(19) United States
(12) Patent Application Publication
(10) Pub. No.: US 2011/0040179 A1
(43) Pub. Date: Feb. 17, 2011

(54) Adaptively Setting A Transmit Frequency In An Ultrasound System

(75) Inventors: Dong Kuk SHIN, Seoul (KR); Jong Sik Kim, Seoul (KR)

Correspondence Address: JONES DAY, 222 EAST 41ST ST, NEW YORK, NY, 10017, US

(73) Assignee: Medison Co., Ltd.

(21) Appl. No.: 12/761,203

(22) Filed: Apr. 15, 2010

(30) Foreign Application Priority Data
Aug. 17, 2009 (KR) 10-2009-0075724

(51) Int. Cl. A61B 8/00

(52) U.S. Cl. 600/437

(57) ABSTRACT

Embodiments for adaptively setting a transmit frequency are disclosed. In one embodiment, by way of non-limiting example, an ultrasound system comprises: a storage unit for storing a mapping table including transmit (Tx) frequencies and scan conditions; and an ultrasound data acquisition unit in communication with the storage unit and being configured to retrieve a Tx frequency corresponding to a scan condition from the storage unit and form ultrasound signals having the retrieved Tx frequency.

### CROSS-REFERENCE TO RELATED APPLICATIONS

The present application claims priority from Korean Patent Application No. 10-2009-0075724 filed on Aug. 17, 2009, the entire subject matter of which is incorporated herein by reference.

### TECHNICAL FIELD

The present disclosure generally relates to ultrasound systems, and more particularly to adaptively setting a transmit frequency in an ultrasound system.

### BACKGROUND

An ultrasound system has become an important and popular diagnostic tool since it has a wide range of applications. Specifically, due to its non-invasive and non-destructive nature, the ultrasound system has been extensively used in the medical profession. Modern high-performance ultrasound systems and techniques are commonly used to produce two or three-dimensional diagnostic images of internal features of a target object (e.g., human organs).

The ultrasound system can transmit and receive ultrasound signals to and from a target object to thereby form 2D (two-dimensional) or 3D (three-dimensional) ultrasound images of the target object. Also, the ultrasound system can form an ultrasound image of the target object by using the receive signals relating to an image depth of the target object and a steering angle of scan-lines. The image depth represents the range of an ultrasound image of the target object, which is to be formed in a depth direction.

### SUMMARY

Embodiments for adaptively setting a transmit frequency in an ultrasound system are disclosed herein. In one embodiment, by way of non-limiting example, an ultrasound system comprises: a storage unit for storing a mapping table including transmit (Tx) frequencies and scan conditions; and an ultrasound data acquisition unit in communication with the storage unit and being configured to retrieve a Tx frequency corresponding to a scan condition from the storage unit and form ultrasound signals having the retrieved Tx frequency.

In another embodiment, there is provided a method of setting a transmit frequency, comprising: a) retrieving a Tx frequency corresponding to a scan condition from a mapping table including transmit (Tx) frequencies and scan conditions; and b) forming ultrasound signals having the retrieved Tx frequency.

In yet another embodiment, there is provided a computer readable medium comprising computer executable instructions configured to perform the following acts: a) storing a mapping table of transmit (Tx) frequencies and scan conditions; b) retrieving a Tx frequency corresponding to a scan condition from the mapping table; and c) forming ultrasound signals having the retrieved Tx frequency.

The Summary is provided to introduce a selection of concepts in a simplified form that are further described below in the Detailed Description. This Summary is not intended to identify key or essential features of the claimed subject matter, nor is it intended to be used in determining the scope of the claimed subject matter.

### BRIEF DESCRIPTION OF THE DRAWINGS

FIG. 1 is a block diagram showing an illustrative embodiment of an ultrasound system.

FIG. 2 is a graph showing an example of a grating lobe according to transmit (Tx) frequencies.

FIG. 3 is a graph showing an example of optimal Tx frequencies set according to image depths.

FIG. 4 is a schematic diagram showing an example of steering angles set according to the image depths.

FIG. 5 is a graph showing an example of optimal Tx frequencies set according to steering angles.

FIG. 6 is a schematic diagram showing an example of the optimal Tx frequencies set according to the image depths and steering angles.

FIG. 7 is a block diagram showing an illustrative embodiment of an ultrasound data acquisition unit.

### DETAILED DESCRIPTION

A detailed description may be provided with reference to the accompanying drawings. One of ordinary skill in the art may realize that the following description is illustrative only and is not in any way limiting. Other embodiments of the present invention may readily suggest themselves to such skilled persons having the benefit of this disclosure.

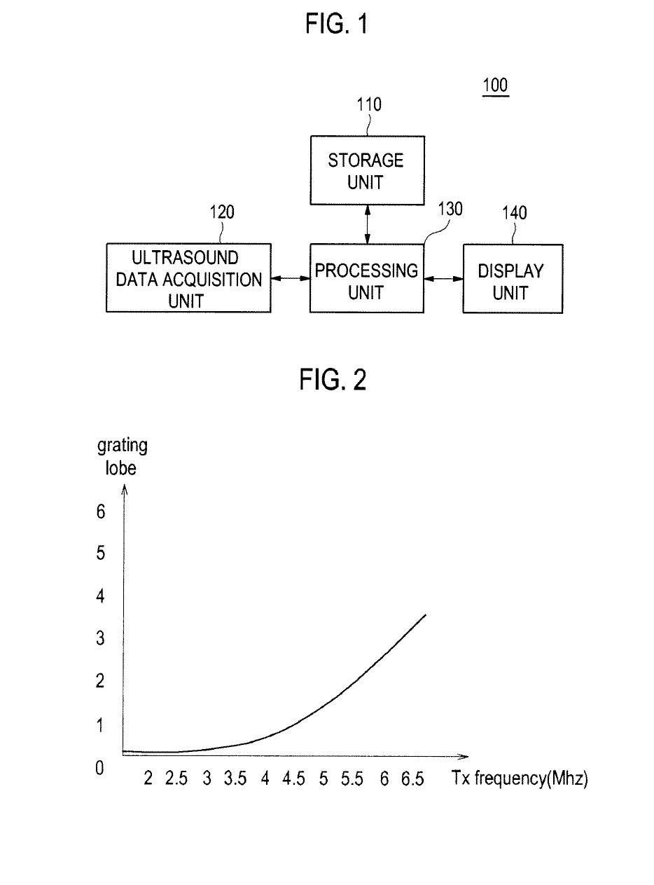

Referring to FIG. 1, an ultrasound system 100 in accordance with an illustrative embodiment is shown. As depicted therein, the ultrasound system 100 may include a storage unit 110. The storage unit 110 may store a mapping table including transmit (Tx) frequencies and scan conditions. In one embodiment, the scan conditions may include image depths for representing the range of an ultrasound image of the target object to be formed in a depth direction and steering angles for steering a plurality of scan-lines. However, the scan conditions may not be limited thereto.

FIG. 2 is a graph showing an example of a grating lobe according to the transmit (Tx) frequencies. Generally, a grating lobe may mean unwanted emission of ultrasound from electronic array transducers at an angle from the main beam. The grating lobe may be stronger as the Tx frequency becomes higher, as shown in FIG. 2. Further, the grating lobe may be stronger as the steering angle becomes larger (not shown).

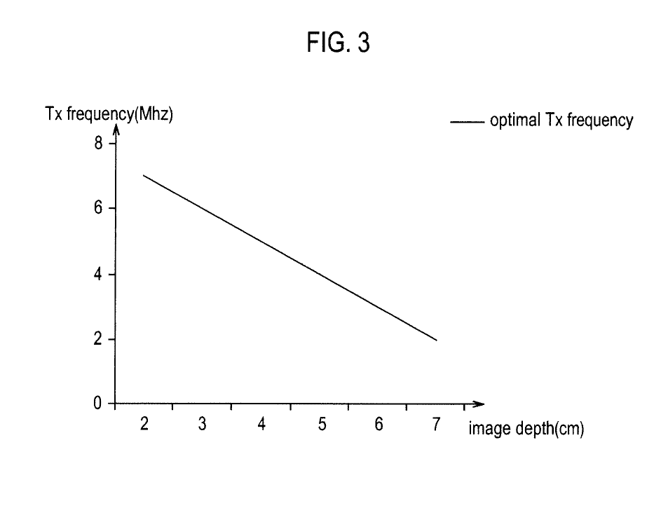

FIG. 3 is a graph showing an example of optimal Tx frequencies according to image depths. As shown in FIG. 3, a higher Tx frequency may be set as the optimal Tx frequency when an image depth is smaller. Also, a lower Tx frequency may be set as the optimal frequency when the image depth is larger.

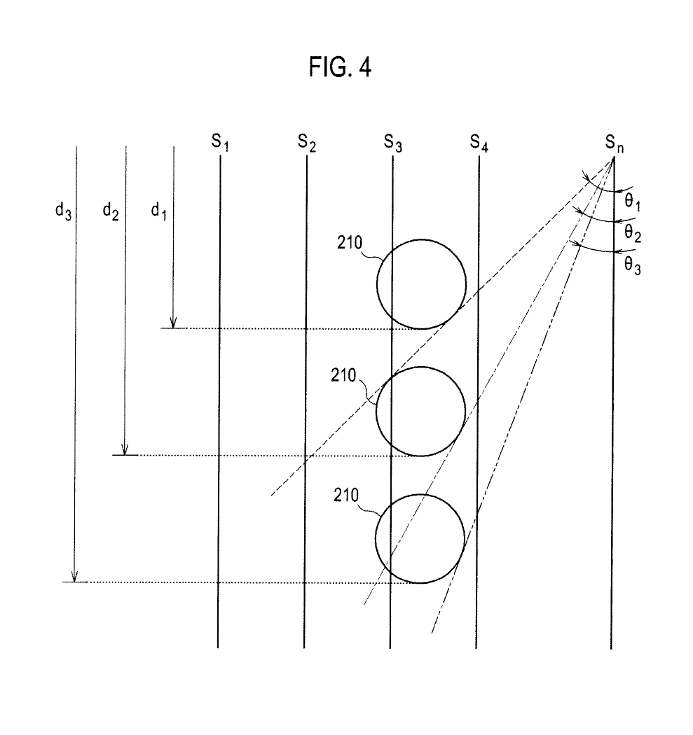

FIG. 4 is a schematic diagram showing an example of steering angles set according to the image depths. As shown in FIG. 4, a larger steering angle may be set when the image depth is smaller. Also, a smaller steering angle may be set when the image depth is larger. That is, steering angles θ₁ to θ₃ of scan-lines S₁ to Sₙ may be set in consideration of image depths d₁ to d₃ for a target object 210, as shown in FIG. 4.

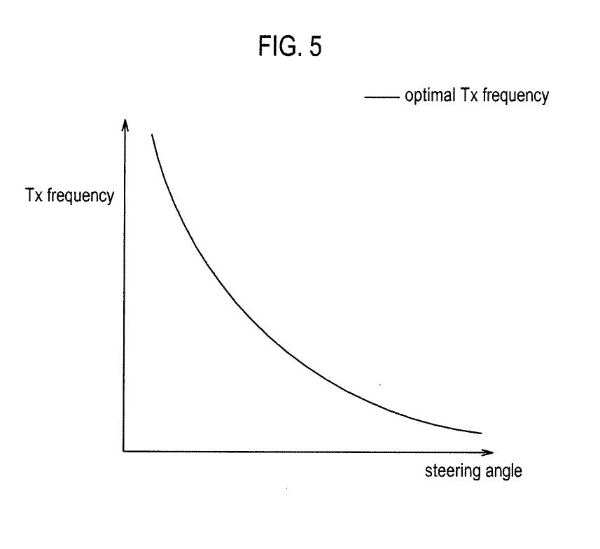

FIG. 5 is a graph showing an example of optimal Tx frequencies set according to the steering angles. As shown in FIG. 5, a lower Tx frequency may be set as the optimal Tx frequency to decrease the grating lobe when a steering angle is larger. Also, a higher Tx frequency may be set as the optimal Tx frequency to decrease the grating lobe when the steering angle is smaller, as shown in FIG. 5.

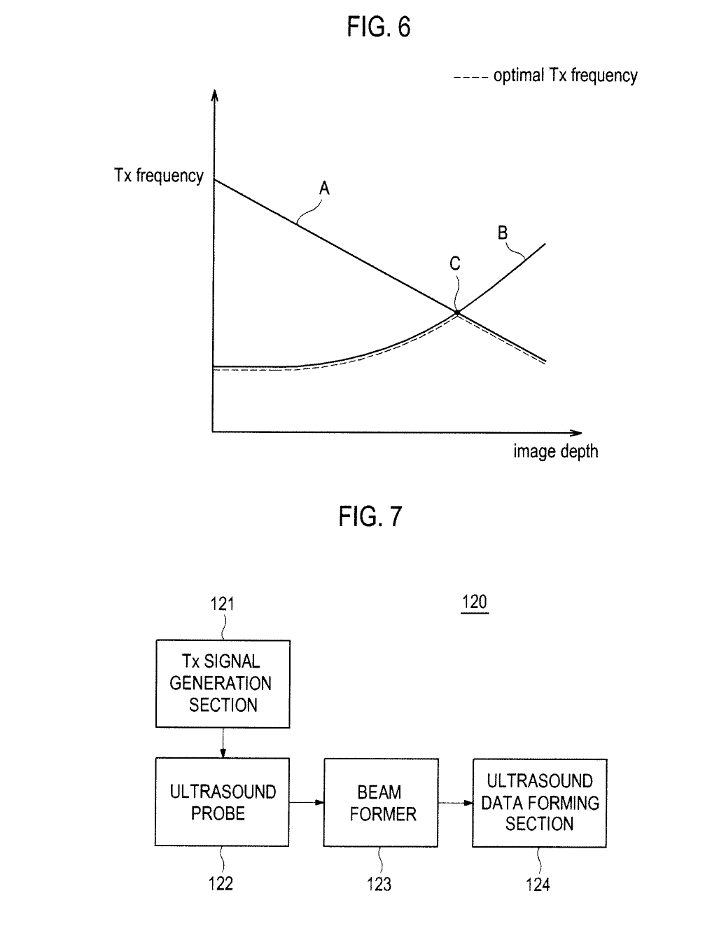

FIG. 6 is a schematic diagram showing an example of the optimal Tx frequencies according to scan conditions (i.e., the image depths and steering angles). When only an image depth for a target object is considered to set an optimal Tx frequency as in a conventional method, the resultant values may be obtained as indicated in A in FIG. 6. Also, when only a steering angle according to the image depth is considered to set an optimal Tx frequency as in the conventional method, the resultant values may be obtained as indicated in B in FIG. 6. However, in this embodiment, both the image depth and the steering angle are considered to set an optimal Tx frequency.

When an image depth for the target object is less than a predetermined threshold value C (i.e., the target object is located on a near position from a surface of a subject, e.g., a patient), the optimal Tx frequency is set according to the steering angle as shown in FIG. 6 to decrease the grating lobe. Also, when the image depth for the target object is more than the predetermined threshold value C (i.e., the target object is located on a far position from the surface of the subject), the optimal Tx frequency is set according to the image depth as shown in FIG. 6 to decrease the grating lobe.

In one embodiment, the mapping table is a table for setting the Tx frequency in consideration of the steering angles when the image depth is less than the predetermined threshold value and setting the Tx frequency in consideration of the image depth when the image depth is more than the predetermined threshold value.

Referring back to FIG. 1, the ultrasound system may further include an ultrasound data acquisition unit 120. The ultrasound data acquisition unit 120 may be configured to transmit and receive ultrasound signals to and from a target object to thereby output ultrasound data.

FIG. 7 is a block diagram showing an illustrative embodiment of the ultrasound data acquisition unit 120. Referring to FIG. 7, the ultrasound data acquisition unit 120 may include a Tx signal generating section 121, an ultrasound probe 122, a beam former 123 and an ultrasound data forming section 124.

The Tx signal generating section 121 may be configured to retrieve a Tx frequency corresponding to a scan condition (i.e., the image depth and steering angle) from the storage unit 110. In one embodiment, the scan condition may be manually set by a user. In another embodiment, the scan condition may be automatically set by the ultrasound system 100. The Tx signal generating section 121 may be further configured to form Tx signals having the retrieved Tx frequency.

The ultrasound probe 122 may include a plurality of elements for reciprocally converting between ultrasound signals and electrical signals. The ultrasound probe 122 may be configured to form ultrasound signals having the retrieved Tx frequency in response to the Tx signals to thereby transmit the ultrasound signals into the target object. The ultrasound probe 122 may further receive echo signals reflected from the target object to thereby output received signals. The received signals may be analog signals. The ultrasound probe 122 may include a linear probe, a convex probe, a 3D (three-dimensional) mechanical probe, a 2D (two-dimensional) probe or the like. However, it should be noted herein that the ultrasound probe 122 may not be limited thereto.

The beam former 123 may be configured to convert the received signals into digital signals. The beam former 123 may further perform receiving-focusing upon the digital signals in consideration of distances between the elements and focal points, the image depth and the steering angle to thereby form digital receive-focused signals.

The ultrasound data forming section 124 may be configured to form ultrasound data based on the digital receive-focused signals. The ultrasound data forming section 124 may be further configured to perform various signal processing (e.g., gain adjustment or the like) to the digital receive-focused signals.

Referring back to FIG. 1, the ultrasound system may further include a processing unit 130. The processing unit 130 may form an ultrasound image of the target object based on the ultrasound data provided from the ultrasound data acquisition unit 120. The ultrasound image may include a brightness mode image, an elastic image, an ultrasound spatial compound image, a 3D (three-dimensional) ultrasound image or the like. However, the ultrasound image may not be limited thereto.

The ultrasound system may further include a display unit 140. The display unit 140 may display the ultrasound image provided from the processing unit 130.

In another embodiment, the present invention may provide a computer readable medium comprising computer executable instructions configured to perform following acts: a) retrieving a Tx frequency corresponding to a scan condition from a mapping table including transmit (Tx) frequencies and scan conditions; and b) forming ultrasound signals having the retrieved Tx frequency. The computer readable medium may comprise a floppy disk, a hard disk, a memory, a compact disk, a digital video disk, etc.

Although embodiments have been described with reference to a number of illustrative embodiments thereof, it should be understood that numerous other modifications and embodiments can be devised by those skilled in the art that will fall within the spirit and scope of the principles of this disclosure. More particularly, numerous variations and modifications are possible in the component parts and/or arrangements of the subject combination arrangement within the scope of the disclosure, the drawings and the appended claims. In addition to variations and modifications in the component parts and/or arrangements, alternative uses will also be apparent to those skilled in the art.

What is claimed is:

1. An ultrasound system, comprising:
a storage unit for storing a mapping table including transmit (Tx) frequencies and scan conditions; and
an ultrasound data acquisition unit in communication with the storage unit and being configured to retrieve a Tx frequency corresponding to a scan condition from the storage unit and form ultrasound signals having the retrieved Tx frequency.

2. The ultrasound system of claim 1, wherein the scan conditions include image depths and steering angles.

3. The ultrasound system of claim 2, wherein the mapping table is a table for setting the Tx frequency in consideration of the steering angles when an image depth of the scan condition is less than a predetermined threshold value and setting the Tx frequency in consideration of the image depths when the image depth of the scan condition is more than the predetermined threshold value.

4. A method of setting a transmit frequency, comprising:
a) retrieving a Tx frequency corresponding to a scan condition from a mapping table including transmit (Tx) frequencies and scan conditions; and
b) forming ultrasound signals having the retrieved Tx frequency.

5. The method of claim 4, wherein the scan conditions include image depths and steering angles.

6. The method of claim 5, wherein the mapping table is a table for setting the Tx frequency in consideration of the steering angles when an image depth of the scan condition is less than a predetermined threshold value and setting the Tx frequency in consideration of the image depth when the image depth of the scan condition is more than the predetermined threshold value.

7. A computer readable medium comprising computer executable instructions configured to perform following acts:
a) retrieving a Tx frequency corresponding to a scan condition from a mapping table including transmit (Tx) frequencies and scan conditions; and
b) forming ultrasound signals having the retrieved Tx frequency.

8. The computer readable medium of claim 7, wherein the scan conditions include image depths and steering angles.

9. The computer readable medium of claim 7, wherein the mapping table is a table for setting the Tx frequency in consideration of the steering angles when an image depth of the scan condition is less than a predetermined threshold value and setting the Tx frequency in consideration of the image depths when the image depth of the scan condition is more than the predetermined threshold value.
